(19) United States
(12) Patent Application Publication
(10) Pub. No.: US 2023/0248588 A1
(43) Pub. Date: Aug. 10, 2023

(54) ABSORBENT PAD COMRPISING COATED SUPERABSORBENT PARTICLES

(71) Applicant: Essity Hygiene and Health Aktiebolag, Goteborg (SE)

(72) Inventor: David Lindqvist, Goteborg (SE)

(73) Assignees: Essity Hygiene and Health Aktiebolag, Goteborg (SE); Essity Hygiene and Health Aktiebolag, Goteborg (SE)

(21) Appl. No.: 17/768,218

(22) PCT Filed: Nov. 12, 2019
(86) PCT No.: PCT/SE2019/051138

§ 371 Date: Apr. 12, 2022

(51) Int. Cl. A61F 13/53; A61F 13/84; A61F 13/15; B01J 20/18

(52) U.S. Cl. CPC A61F 13/53; A61F 13/8405; A61F 13/15658; B01J 20/18; A61F 2013/530496; A61F 2013/8408

(57) ABSTRACT

An absorbent pad, such as a feminine hygiene article, according to the present disclosure comprises superabsorbent polymer particles (2). The superabsorbent polymer particles (2) are surface coated on an outer surface (3) with a coating agent (4), the coating agent (4) being water-soluble. The coated superabsorbent polymer particles (2) comprises an odour inhibiting substance (5) in powder form adhered to and/or mixed with the coating agent (3). The present disclosure also relates to methods of producing the absorbent pad (1).

### CROSS REFERENCE TO RELATED APPLICATIONS

This application is a National Stage application of PCT/SE2019/051138, filed Nov. 12, 2019, which is incorporated by reference in its entirety herein.

### TECHNICAL FIELD

The present disclosure relates to an absorbent pad comprising an odour inhibiting substance in powder form. The present disclosure furthermore relates to methods of producing an absorbent pad comprising an odour inhibiting substance in powder form.

### BACKGROUND

Absorbent pads of the kind to which this disclosure relates are worn against the skin with a topsheet facing the skin of the user and are provided with an absorbent core for absorbing bodily fluids of the wearer. Bodily fluids, such as blood and urine, are collected and stored in the absorbent products and odours may easily arise. These odours can be embarrassing for the wearer of the article. It is important, therefore, to reduce or prevent odours from occurring in absorbent pads while they are being worn.

In the field of absorbent pads, several different solutions are used to prevent odours. For example, odours can be masked by the use of perfumes or deodorizing compounds. Odours may also be adsorbed or absorbed to particles having a large surface area, such as activated carbon, zeolite and starch-based particulate materials. Acidic and/or alkaline odours may be neutralized by the use of substances like baking soda and/or citric acid. For bacteria inhibition, substances having low pH or metal salts can be used. Accordingly, different odour control agents may be used to prevent odours in different manners.

Odour control agents in particulate form, such as the above-mentioned activated carbon, zeolite and starch-based materials, have been proved to have excellent odour-adsorbing characteristics due to the large surface area of the particles. For odour control agent in powder form, such as zeolite powder, there is however some difficulties connected to the use of such in absorbent pads and the handling of the powder in the absorbent article manufacturing. Powders may contaminate both process equipment and products.

An aim of the present invention is to provide an improved absorbent pad comprising an odour control agent in powder form which is efficient and easy to implement in absorbent pads.

### SUMMARY

One or more of the above objects may be achieved with an absorbent pad in accordance with claim 1 and methods in accordance with claims 16 and 17. Further embodiments are set out in the dependent claims, in the following description and in the drawings.

The absorbent pad as disclosed herein comprises superabsorbent polymer particles. The superabsorbent polymer particles are surface coated on an outer surface with a coating agent being water-soluble. The coated superabsorbent polymer particles furthermore comprise an odour inhibiting substance in powder form adhered to and/or mixed with the coating agent.

The term “absorbent pad” refers to products that are placed against the skin of the wearer to absorb and contain body exudates, like urine, feces and menstrual fluid. The invention refers to disposable absorbent pads, which means articles that are not intended to be laundered or otherwise restored or reused as a sanitary article. Examples of disposable absorbent pads include feminine hygiene products such as sanitary napkins, panty liners, sanitary panties and feminine inserts; diapers for infants and incontinent adults; incontinence pads; diaper inserts and the like.

“Superabsorbent polymer particles” (SAP) as used herein, refers to particles absorbing at least 10 grams of 0.9% saline solution per gram of the superabsorbent polymer particle. The superabsorbent polymer may for example be a polyacrylic acid-based SAP.

By “water-soluble” herein is meant that the coating agent is able to be dissolved in water having a temperature of from 23° C.

Some efficient odour inhibiting substances are provided in the form of very small dusty particles which have been found difficult to handle in the manufacturing of absorbent articles as the powder tend to be blown or vacuumed up from absorbent structures moving at high speed during manufacturing of absorbent pad. The present inventors have found that by using superabsorbent particles as a carrier and by providing a water-soluble coating agent to which the odour control substances in powder form may be adhered, the odour control substances may be provided in an absorbent pad without risking to compromise the machinery and without any risk of impairing the liquid absorption function of the superabsorbent polymer particles.

The odour inhibiting substance in powder form may be in the form of powder particles, the powder particles having an average particle size within the range of from 0.5 μm to 50 μm. The average particle size being measured according to the method ISO 13322-1:2014, PARTICLE SIZE ANALYSIS—IMAGE ANALYSIS METHODS—PART 1: STATIC IMAGE ANALYSIS METHODS.

ISO 13322-1:2014 is applicable to the analysis of images for the purpose of determining particle size distributions where the velocity of the particles against an axis of the optical system of the imaging device is zero. The particles are appropriately dispersed and fixed in the object plane of the instrument. The field of view may sample the object plane dynamically either by moving the sample support or the camera provided this can be accomplished without any motion effects on the image. Captured images can be analysed subsequently. ISO 13322-1:2014 concentrates upon the analysis of digital images created from either light or electron detection systems. It considers only image evaluation methods using complete pixel counts.

The absorbent pad may comprise an amount of from 0.1 g to 20 g of the superabsorbent polymer particles.

The odour inhibiting substance in powder form may be a silicate powder, such as an aluminosilicate mineral powder. The SiO:Al ratio of the silicate powder may be from 10 to 1000. The SiO:Al ratio of the silicate powder may optionally be from 350 to 1000. Such odour inhibiting substances with a low amount of aluminium have been found particularly efficient on malodours connected to feminine sanitary pads.

The silicate powder may be zeolite powder. The SiO:Al ratio of the zeolite powder may be from 10. Zeolite powder having an SiO:Al ratio of from 10 to 1000, such as from 350 to 1000, have been found particularly effective in inhibiting a wide range of malodours often occurring during use of feminine sanitary pads, such as diacetyl. 3-methyl-butanal and dimethyl disulphide (DMDS).

The coating may be adhesively adhered to the outer surface of the superabsorbent polymer particle. The odour inhibiting substances in powder may be both mixed with the coating agent and/or applied to the outer surface of the superabsorbent polymer particles provided with an outer layer of the coating agent.

The coating agent may be in a liquid state at 23° C.

The coating agent is water-soluble upon contact of the surface coated superabsorbent polymer particles with an aqueous liquid, such as a bodily fluid. Thus, when the superabsorbent polymer particles are subjected to liquid, a substantive part of the coating is washed off. As the coating agent only adheres to the superabsorbent polymer particles due to its adhesive properties, i.e. not by being bound to the surface of the superabsorbent particles by means of chemical bounds, and as it is water-soluble the coating agent together with the odour inhibiting substance in powder form may be washed away from the superabsorbent polymer particles upon contact between the superabsorbent particles and bodily fluids. Therefore, the absorbent properties of the coated superabsorbent particles according to the present disclosure are not deteriorated due to the coating.

The coating agent being in a liquid state at 23° C. may facilitate dissolving the coating upon wetting of the absorbent pad.

The coating agent may be a polyol, for example a glycerol. To provide the coating agent with a proper viscosity and to increase the amount of odour inhibiting powder, the coating agent may be mixed with the odour inhibiting powder. In a mixture of coating agent and the odour inhibiting powder, the amount of coating agent may be from 5 wt. % to 25 wt. % and the amount of odour inhibiting powder may be from 75 wt. % to 95 wt. %. The odour inhibiting powder may additionally and in a subsequent step be applied as an external powder layer on the coating mixture of the coating agent and the odour inhibiting substance in powder form. Polyols, and particularly glycerol, has been found by the present inventor to have a surprisingly low impact on the odour inhibiting properties of the odour inhibiting substances and particularly so glycerol.

The coated superabsorbent particles may comprise from 2 times, such as from 3 times as much odour inhibiting substance as coating agent. The coated superabsorbent particles may comprise up to 5 times, such as up to 10 times as much odour inhibiting substance as coating agent.

The amount of coating agent based on the weight of the total weight, prior to coating, of the superabsorbent particles may be from 0.5 wt. % to 5 wt. %.

The amount of odour inhibiting substance based on the weight of the total weight of the superabsorbent particles may be from 5 wt. % to 35 wt. %.

The absorbent pad may comprise a topsheet, a backsheet and an absorbent core arranged between the topsheet and the backsheet and wherein the absorbent core includes the superabsorbent particles. The absorbent core may for example be a mix of cellulose fibers and the coated superabsorbent particles according to the present disclosure. The absorbent core may for example comprise an amount of cellulose fibers in particles in the range of from 5 to 45 wt. %.

The method of producing an absorbent pad, such as a feminine hygiene article, comprising superabsorbent polymer particles, the superabsorbent polymer particles being surface coated on an outer surface with a coating agent being water-soluble, as disclosed herein comprises the steps of;
a) providing superabsorbent polymer particles,
b) mixing the superabsorbent polymer particles with the coating agent,
c) applying an odour inhibiting substance in powder form to the surface coated superabsorbent polymer particles, and;
d) forming an absorbent pad and integrating the coated superabsorbent polymer particles into the absorbent pad.

According to a further aspect, the method of producing an absorbent pad, such as a feminine hygiene article, comprising superabsorbent polymer particles, the superabsorbent polymer particles being surface coated on an outer surface with a coating agent being water-soluble, as disclosed herein comprises the steps of;
a) providing superabsorbent polymer particles,
b) mixing the coating agent with an odour inhibiting substance in powder form,
c) mixing the superabsorbent polymer particles with the mixture obtained in step b), optionally applying an outer layer with the odour inhibiting substance in powder form on the coated superabsorbent polymer particles, and;
d) forming an absorbent pad and integrating the coated superabsorbent polymer particles into the absorbent pad.

The coating step(s) may for example be carried out in a fluidized bed coating process. The coating agent may be added in an amount of 0.5 wt. % to 5 wt. % and the amount of odour inhibiting substances may be from 5 wt. % to 35 wt. %, based on the weight of the total weight of the superabsorbent particles.

The odour inhibiting substance in powder form may be a silicate powder, such as an aluminosilicate mineral powder, optionally the silicate powder having SiO:Al ratio of from 10 to 1000.

The silicate powder may be a zeolite powder.

The coating agent may be a polyol, for example glycerol.

The coating agent may be a coating agent being in a liquid state at 23° C. The coating agent being in a liquid state at 23° C. may simplify the coating step.

The absorbent pad may comprises a topsheet, a backsheet and an absorbent core arranged between the topsheet and the backsheet and wherein the method step d) include to incorporate the coated superabsorbent particles in the absorbent core.

The absorbent core may include a mixture of cellulose fibers and the coated superabsorbent particles.

### BRIEF DESCRIPTION OF THE DRAWINGS

The present invention will be further explained hereinafter by means of non-limiting examples and with reference to the appended drawings wherein:

FIG. 1 illustrates a superabsorbent particle according to the present disclosure;

FIG. 2 illustrates a fibrous network including the superabsorbent polymer particles;

FIG. 3 shows a top plan view of an absorbent pad;

FIG. 4 shows a bar diagram illustrating the results of an odour inhibition test between absorbent pads according to the present disclosure and reference samples; and

FIG. 5 shows a bar diagram illustrating the results from an odour inhibition test of absorbent core samples comprising coated superabsorbent polymer particles according the present disclosure and compared with reference absorbent core samples comprising 20 mg of zeolite powder.

### DETAILED DESCRIPTION

The invention will be described more closely below by reference to an exemplary embodiment. The invention may however be embodied in many different forms and should not be construed as limited to the embodiments set forth in the drawings and the description thereto.

FIG. 1 schematically shows a coated superabsorbent polymer particle 2 according to the present disclosure. The superabsorbent polymer particle 2 is coated with a coating agent 4 being water-soluble and in a liquid form at 23° C., such as glycerol, on an outer surface 3 thereof. The coating agent 4 is provided as a surface-coating and is adhesively adhered to the outer surface 3 of the superabsorbent polymer particle 2. The coating agent 4 is mixed with the odour inhibiting substance 5 in powder form, such as a zeolite powder, to provide the coating agent 4 with a suitable viscosity and stickiness for adhering well to the superabsorbent particles 2. A mixture of the coating agent 4 and the odour inhibiting substance in powder form 5 may for example comprise coating agent 4 in an amount of 5 to 25% and the odour inhibiting substance in powder form 5 in an amount of 75% to 95%. As an outermost layer, a powder layer of the odour inhibiting substances in powder form 5 has been applied on the sticky coating layer including the mixture of the coating agent 4 and the odour inhibiting substance in powder form 5. The superabsorbent particles may be polyacryl-based superabsorbent particles.

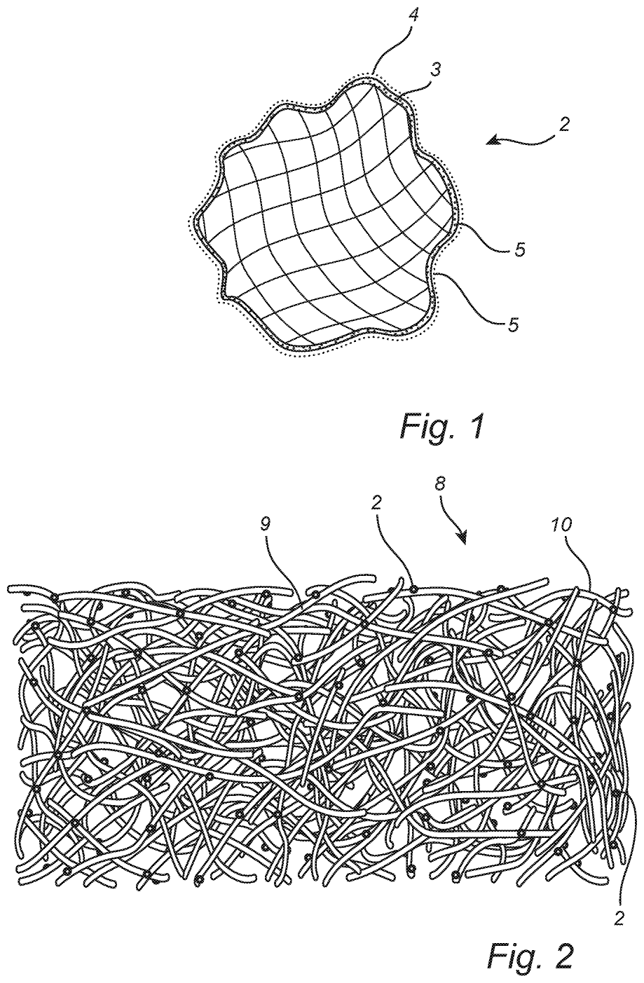

FIG. 2 illustrates a portion of an absorbent core 8, the absorbent core 8 including a fibrous structure 9 comprising a network of cellulosic fibers 10 mixed with coated superabsorbent polymer particles 2. The absorbent core 8 may include from 0.1 grams to 20 grams of the coated superabsorbent polymer particles 2. As the coating agent is water-soluble, the coating may upon contact with bodily fluid dissolve from the superabsorbent particles and the slippery surface of the superabsorbent particles upon wetting and the absorbent properties of the superabsorbent particles may remain unaffected. The odour inhibiting effect of the odour inhibiting substance in powder form will also be preserved, as the odour inhibiting substance together with the coating agent will remain in the fiber network.

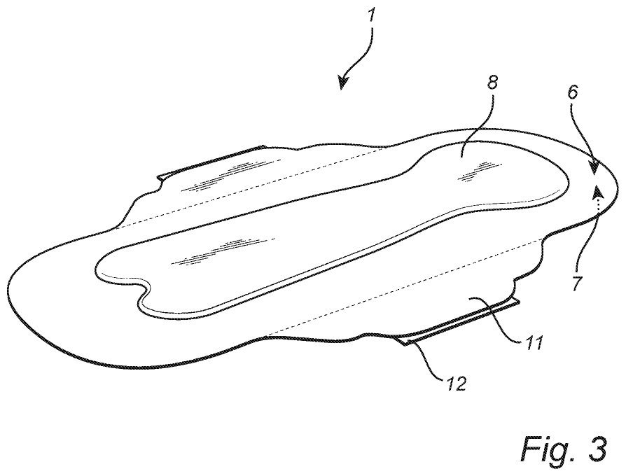

FIG. 3 schematically shows an absorbent pad 1 according to the present disclosure, the absorbent pad 1 being in the form of a sanitary napkin 1. The sanitary napkin 1 comprises a fluid permeable topsheet 6, a backsheet 7 and an absorbent core 8 interposed between the topsheet 6 and the backsheet 7. The absorbent pad 1 is provided with laterally extending flaps 11 being provided with attachment means 12 on a garment facing side. The backsheet 7 may be provided with an adhesive provided on an outwardly oriented side for attachment of the sanitary article 1 to an undergarment on a user-facing side of the undergarment.

The topsheet may be a plastic perforated film and/or may include or consist of fibrous nonwoven layer(s). The fibrous nonwoven layers may be spunbonded, meltblown, carded, hydroentangled, wetlaid etc. Suitable nonwoven materials can be composed of natural fibers, such as woodpulp or cotton fibres, synthetic thermoplastic fibres, such as polyolefins, polyesters, polyamides and blends and combinations thereof or from a mixture of natural and synthetic fibres. The materials suited as topsheet materials should be soft and non-irritating to the skin and be readily penetrated by body fluid, such as urine or menstrual fluid. The topsheet material may essentially consist of synthetic thermoplastic fibers, such as polyolefins, polyesters, polyamides and blends and combinations thereof. The synthetic fibers may be monocomponent fibers, bicomponent fibers or multicomponent fibers including polyesters, polyamides and/or polyolefins such as polypropylene and polyethylene.

The absorbent core may be of any conventional kind. In addition to superabsorbent polymer particles, examples of commonly occurring absorbent materials are cellulosic fluff pulp, tissue layers, absorbent foam materials, absorbent nonwoven materials or the like. It is common to combine cellulosic fluff pulp with superabsorbent polymer particles in an absorbent structure. It is also common to have absorbent structures comprising layers of different material with different properties with respect to liquid acquisition capacity, liquid distribution capacity and storage capacity. This is well-known to the person skilled in the art and does therefore not have to be described in detail. The thin absorbent bodies, which are common in today's sanitary articles, often comprise a compressed mixed or layered structure of cellulosic fluff pulp and superabsorbent. The size and absorbent capacity of the absorbent structure may be varied to be suited for different uses such as sanitary articles, pantyliners, adult incontinence pads and diapers, baby diapers, pant diapers, etc.

The backsheet may consist of a thin plastic film, e.g. a polyethylene or polypropylene film, a nonwoven material coated with a liquid impervious material, a hydrophobic nonwoven material, which resists liquid penetration. Laminates of plastic films and nonwoven materials may also be used. The backsheet material can be breathable so as to allow vapor to escape from the absorbent structure, while still preventing liquids from passing through the backsheet material.

### Experimental Section

### Absorbent Capacity Test

In order to evaluate the impact of the coating on the absorbency properties of the superabsorbent polymer particles according to the present disclosure, the free swelling and retention capacity of three different samples of coated superabsorbent particles were prepared, evaluated and compared with a reference sample of a superabsorbent polymer particle coated only with a zeolite powder using the methods Free Swelling Capacity (FSC) as described in the standard method NWSP 240.0 (R2) and Centrifuge Retention Capacity (CRC) as described in the standard method NWSP 241.0 (R2).

The coating agents tested were glycerol and two different polyethylene glycols (PEG); PEG1000 and PEG300. PEG 300 is a polyethylene glycol having an average molecular weight of 300 g/mol and PEG 1000 is a polyethylene glycol having an average molecular weight of 1000 g/mol.

The coating agent were added to the superabsorbent particles (Favor SXM 9420 from Evonik) in a liquid form, which means that the PEG 1000 was heated to 50° C. before it was added to the superabsorbent particles. In a subsequent step the superabsorbent polymer particles coated with the coating agent were mixed with zeolite powder (ZEOflair 100 from Zeochem AG) and a small amount of active carbon. A mix of zeolite powder and active carbon powder was thus added to the different samples of the coated superabsorbent polymer particles, the zeolite powder in an amount of 10 wt. %, of the amount of superabsorbent polymer particles, and 0.1 g active carbon. The active carbon was included in order to enable visual verification that the powder mix of zeolite and active carbon was evenly distributed onto the superabsorbent particles. The glycerol and the PEG 300 was added in an amount of 1.75 wt. %, of the amount of superabsorbent polymer, and PEG 1000 was added in an amount of 2.75 wt. %, of the amount of superabsorbent polymer.

The Free Swelling Capacity of the coated superabsorbent polymer particles were measured after 1 minute, after 3 minutes and after 30 minutes and the Centrifuge Retention Capacity of the coated superabsorbent polymer particles was also measured. Reference values obtained from the superabsorbent polymer particle manufacturer are included in the tables.

The superabsorbent particles in table 1 are reference samples and are covered with 10 wt. % zeolite powder, based on the weight of the superabsorbent particles, and 0.1 g active carbon. Values adjusted for the additional 10 wt. % of zeolite powder included are also shown below.

TABLE 1

| | | FSC | FSC | FSC | CRC | CRC |
|---|---|---|---|---|---|---|
| | | 1 min | 5 min | 30 min | g/g | % |
| Ref SXM9420 + | Average g/g | 19 | 41 | 51 | 30 | 59 |
| 10% Zeolite and | Std | 1.1 | 1.0 | 0.7 | 0.2 | 0.7 |
| 0.1 g AC | Adjusted for | 20.7 | 46.0 | 56.3 | 33 | |
| | 10% zeolite | | | | | |
| 20180702-006 | Ref. | 20 | 48 | 58 | 34 | 58 |

As seen in table 1, the free swelling capacity and the centrifuge retention capacity of the superabsorbent polymer particles were in principle unaffected by the addition of zeolite powder.

In table 2 results from tests on superabsorbent polymer particles coated with 1.75 wt. % PEG300, 10 wt. % zeolite powder and 0.1 g active carbon are illustrated. Values adjusted for the additional 10 wt. % of zeolite powder included are also shown below.

TABLE 2

| | | FSC | FSC | FSC | CRC | CRC |
|---|---|---|---|---|---|---|
| | | 1 min | 5 min | 30 min | g/g | % |
| Ref SXM9420 | Average | 19 | 40 | 50 | 30 | 59 |
| 1.75% PEG300 | g/g | | | | | |
| 10% zeolite and | Std | 0.5 | 0.8 | 0.5 | 0.1 | 0.6 |
| 0.1 g AC | Adjusted for | 20.6 | 44.9 | 55.5 | 33 | 60 |
| | 10% zeolite | | | | | |
| 20180702-006 | Ref. | 20 | 48 | 58 | 34 | 58 |

As illustrated in table 2, there is no significant impact of the coating of the superabsorbent polymer particles on the free swelling capacity and the centrifuge retention capacity of the superabsorbent polymer particles coated with PEG 300 and zeolite powder.

In table 3 results from tests on superabsorbent polymer particles coated with 1.75 wt. % Glycerol, 10 wt. % zeolite and 0.1 g active carbon are shown.

TABLE 3

| | | FSC | FSC | FSC | CRC | CRC |
|---|---|---|---|---|---|---|
| | | 1 min | 5 min | 30 min | g/g | % |
| SXM9420 | Average | 16 | 40 | 49 | 30 | 60 |
| 1.75% Glycerol | g/g | | | | | |
| 10% Zeolit and | Std | 0.3 | 0.7 | 1.4 | 0.2 | 1.7 |
| 0.1 g AC | Adjusted for | 18.0 | 44.0 | 54.8 | 33 | 60 |
| | 10% zeolite | | | | | |
| 20180702-006 | Ref. | 20 | 48 | 58 | 34 | 58 |

As illustrated in table 3, there is no significant impact of the coating of the superabsorbent polymer particles on the free swelling capacity and the centrifuge retention capacity on the superabsorbent polymer particles coated with glycerol and zeolite powder.

In table 4 the results from tests made on superabsorbent polymer particles coated with 2.75% PEG, 10% zeolite and 0.1 g AC are shown.

TABLE 4

| | | FSC | FSC | FSC | CRC | CRC |
|---|---|---|---|---|---|---|
| | | 1 min | 5 min | 30 min | g/g | % |
| Ref SXM9420 + 10% | Average | 19 | 42 | 50 | 29 | 58 |
| Zeolite + 2.75% | g/g | | | | | |
| PEG1000, | Std | 1.1 | 0.9 | 0.4 | 0.5 | 0.5 |
| and 0.1 g AC | Adjusted for | 20.7 | 46.5 | 55.3 | 33 | 60 |
| | 10% zeolite | | | | | |
| | Ref. | 20.0 | 47.0 | 57.0 | 34 | 58 |

As illustrated in table 4, there is no significant impact of the coating of the superabsorbent polymer particles on the free swelling capacity and the centrifuge retention capacity on the superabsorbent polymer particles coated with PEG 1000 and zeolite powder.

### Odour Inhibition Tests

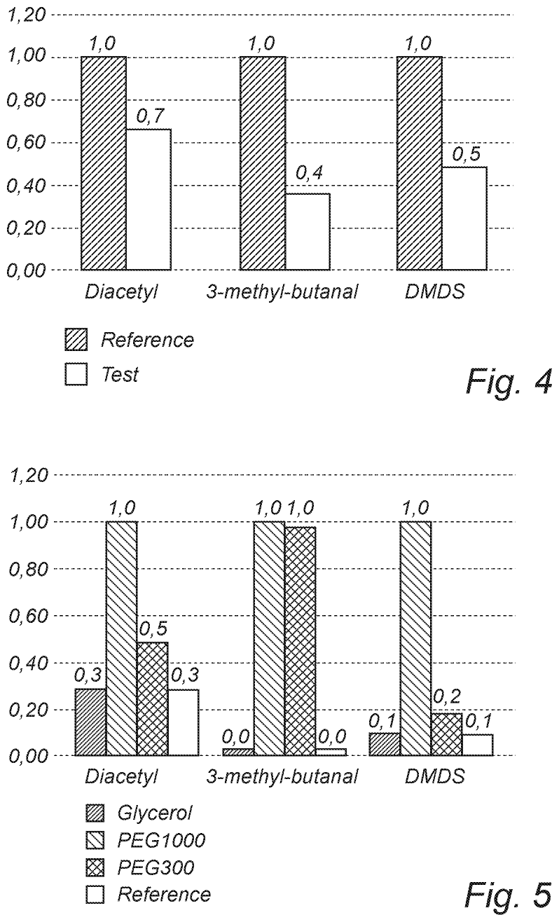

Measurements were performed to evaluate the odour inhibition efficiency in an absorbent pad according to two different methods illustrated in FIGS. 4 and 5. A first method IMOR Feminine Between was carried out at RISE—Research Institute of Sweden, in Gothenburg, Sweden and a second method RO-liners was carried out at Essity Hygiene and Health AB, in Gothenburg, Sweden.

In the respective methods, the test liquid are used to simulate the odour of used feminine liners. The test liquid consists of a buffer solution, pH 4.0, comprising the odorants diacetyl, 3-methylbutanal, dimethyl disulphide (DMDS), 1-octene-3-one and isovaleric acid (IVA).

### IMOR Feminine Between

In a first test, IMOR Feminine Between, illustrated in FIG. 4, a reference liner; Liner Long Protege Slip, Deliplus was compared to a test liner; Liner Long Protege Slip, Deliplus including 20 mg zeolite powder (ZepFlair), 0.2 mg active carbon.

Test Set-Up
2.5 ml of test liquid was added to each sample
The liners were tested in triplicates

### Test Method

As described above, test liquids are used to simulate the odour of used feminine liners. The test liquid consists of a buffer solution, pH 4.0, comprising the odorants diacetyl, 3-methylbutanal, dimethyl disulphide (DMDS), 1-octene-3-one and isovaleric acid (IVA).

To each of the liners 2.5 ml of the test liquid were added, and the wetted samples were equilibrated at 35° C. for 4 hours in a 4.8 l glass vessel (IMOR vessel).

After 4 hours, the odorants remaining in gas phase in the glass vessels are sampled on an adsorbent Tenax tube and samples are taken out from the glass vessels and analysed using Gas Chromatography. The concentration of each odorant is reported as ng/I. The results are compared to those of a reference and are illustrated in FIG. 4.

### RO-Liner

In a further method, odour inhibition of absorbent core samples comprising coated superabsorbent polymer particles according to this disclosure was evaluated and compared with a reference absorbent core samples comprising 20 mg of zeolite powder. The purpose of the test was to investigate any possible negative impact on the odour reducing effect of the zeolite powder when being coated on superabsorbent polymer particles with a coating agent in accordance with the present disclosure. The result being illustrated in FIG. 5.

The coating agents tested were glycerol and two different polyethylene glycols (PEG); PEG1000 and PEG300, wherein PEG 300 is a polyethylene glycol having an average molecular weight of 300 g/mol and PEG 1000 is a polyethylene glycol having an average molecular weight of 1000 g/mol.

The coating agent was added to the superabsorbent particles in a liquid form, which means that the polyethylene glycol 1000 (PEG) was heated to 50° C. before it was added to the superabsorbent particles. In a subsequent step the superabsorbent particle coated with the coating agent were mixed with zeolite powder (ZEOflair 100 from XXXX) mixed with a small amount of active carbon. A mix of zeolite powder and active carbon powder was thus added to the different samples of the coated superabsorbent polymer particles, the zeolite powder in an amount of 10 wt. of the amount of superabsorbent polymer particles, and 0.1 g active carbon. The active carbon was included in order to enable visual verification that the powder mix of zeolite and active carbon was evenly distributed onto the superabsorbent particles. The respective coating agent of the glycerol and the PEG 300 was added in an amount of 1.75 wt. % of the amount of superabsorbent polymer and PEG 1000 was added in an amount of 2.75 wt. % of the amount of superabsorbent polymer.

All samples, including the reference sample, contains 20 mg zeolite powder. The coated superabsorbent particles was included into a cut sample absorbent core having a diameter of 50 mm, the absorbent core comprising cellulose fibers and the coated superabsorbent polymers. The absorbent core was covered with a nonwoven topsheet and sealed with a pre-glued nonwoven covering the back and the sides of the respective sample. The nonwoven material being an 18 gsm hydrophilic spunbond, 100% polypropylene, nonwoven material.

Test Set-Up
2.5 ml of test liquid was added to each sample
The samples were tested in triplicates, one day for each method.

As described above, test liquids are used to simulate the odour of used feminine liners. The test liquid consists of a buffer solution, pH 4.0, comprising the odorants diacetyl, 3-methylbutanal, dimethyl disulphide (DMDS), 1-octene-3-one and isovaleric acid (IVA).

To each of the samples 2.5 ml of the test liquid were added, and the wetted samples were equilibrated at 35° C. for 4 hours in a 500 ml glass bottle (Duran bottle).

During this time, the odour inhibitor reacts with the odorants. After 4 hours, the odorants remaining in gas phase in the glass bottle are sampled on an adsorbent Tenax tube and samples are taken out from the glass bottles and analysed using Gas Chromatography. The concentration of each odorant is reported as ng/I. The results are compared to those of a reference.

In the respective methods, the sample with the least good result was set as the normalized result, i.e. 1.0, and the results of the other samples were compared with this normalized result 1.0.

### Results

In the results from the Feminine in Between method, shown in FIG. 4, it is clearly illustrated that the zeolite powder added to the absorbent core has a significant odour inhibiting effect on the odorants included in the test liquids.

In the RO-Liner method illustrated in FIG. 5, it may be seen that when comparing the coating agents and their possible impact on the odour inhibiting effect of the zeolite powder, glycerol has the overall best performance on each of the three evaluated odours. Glycerol did not at all affect the odour inhibiting effect of the zeolite powder. The coated superabsorbent polymer particles using PEG 300 as coating agent also showed a very low negative impact of the coating agent on the odour inhibiting effect on at least two of the odours, Diacetyl and DMDS.

The coated superabsorbent polymer particles using PEG 1000 as coating agent showed an odour inhibiting effect on each of the odours, but the coating agent PEG 1000 reduced the effect to some degree when comparing with an absorbent core including the same amount of zeolite powder in free form.

1. An absorbent pad, such as a feminine hygiene article, the absorbent pad comprising:
superabsorbent polymer particles, the superabsorbent polymer particles being surface coated on an outer surface with a coating agent, wherein the coating agent is water-soluble and is a polyol, for example glycerol, and wherein the coated superabsorbent polymer particles includes odour inhibiting substances in powder form adhered to and/or mixed with the coating agent.

2. The absorbent pad according to claim 1, wherein the odour inhibiting substances in powder form includes powder particles, the powder particles having an average particle size within the range of from 0.5 μm to 50 μm, the average size particle being measured according to ISO 13322-1:2014.

3. The absorbent pad according to claim 1, wherein the absorbent pad includes an amount of from 0.1 g to 20 g of the coated superabsorbent polymermparticles.

4. The absorbent pad according to claim 1, wherein the odour inhibiting substances in powder form is a silicate powder, such as an aluminosilicate mineral powder.

5. The absorbent pad according to claim 4, wherein the silicate powder is zeolite powder.

6. The absorbent pad according to claim 4, wherein the SiO:Al ratio of the silicate powder is from 10 to 1000.

7. The absorbent pad according to claim 1, wherein the coating agent is adhesively adhered to the outer surface of the superabsorbent polymer particle.

8. The absorbent pad according to claim 1, wherein the coating agent is water-soluble upon contact of the surface coated superabsorbent polymer particles with an aqueous liquid.

9. The absorbent pad according to claim 1, wherein the coating agent is in a liquid state at 23° C.

10. The absorbent pad according to claim 1, wherein the superabsorbent polymer is polyacrylic acid.

11. The absorbent pad according to claim 1, wherein the amount of coating agent based on the weight of the total weight of the superabsorbent polymer particles is from 0.5 wt. % to 5 wt. %.

12. The absorbent pad according to claim 1, wherein the amount of odour inhibiting substances based on the weight of the total weight of the superabsorbent polymer particles is from 5 wt. % to 35 wt. %.

13. The absorbent pad according to claim 1, wherein the absorbent pad comprises a topsheet, a backsheet and an absorbent core arranged between the topsheet and the backsheet and wherein the absorbent core includes the coated superabsorbent particles.

14. The absorbent pad according to claim 13, wherein the absorbent core includes a mixture of cellulose fibers and the coated superabsorbent particles.

15. A method of producing an absorbent pad, such as a feminine hygiene article, including superabsorbent polymer particles (2), the superabsorbent polymer particles being surface coated on an outer surface with a coating agent being water-soluble and is a polyol, for example glycerol, the methods comprising the steps of:
a) providing superabsorbent polymer particles,
b) mixing the superabsorbent polymer particles with the coating agent,
c) applying an odour inhibiting substance in powder form to the surface coated superabsorbent polymer particles, and; d) forming an absorbent pad and integrating the coated superabsorbent polymer particles into the absorbent pad.

16. A method of producing an absorbent pad, such as a feminine hygiene article, including superabsorbent polymer particles, the superabsorbent polymer particles being surface coated on an outer surface with a coating agent being water-soluble and is a polyol, for example glycerol, the method comprising the steps of:
a) providing 5 superabsorbent polymer particles,
b) mixing the coating agent with an odour inhibiting substance in powder form,
c) mixing the superabsorbent polymer particles with the mixture obtained in step b) and;
d) forming an absorbent pad and integrating the coated superabsorbent polymer particles into the absorbent pad.

17. The method according to claim 15, wherein the odour inhibiting substance in powder form is a silicate powder, such as an aluminosilicate mineral powder, optionally the silicate powder having SiO:Al ratio of from 10 to 1000.

18. The method according to claim 17, wherein the silicate powder is a zeolite powder.

19. The method according to claim 15, wherein the coating agent is in a liquid state at 23° C.

20. The method according to claim 15, wherein the absorbent pad comprises a topsheet, a backsheet and an absorbent core arranged between the topsheet and the backsheet and wherein the method step d) include to incorporate the coated superabsorbent particles in the absorbent core.

21. The method according to claim 20, wherein the absorbent core includes a mixture of cellulose fibers and the coated superabsorbent particles.

22. The method according to claim 16, wherein the odor inhibiting substance in powder form is a silicate powder, such as an aluminosilicate mineral powder, optionally the silicate powder having SiO:Al ratio of from 10 to 1000.

23. The method according to claim 22, wherein the silicate powder is a zeolite powder.

24. The method according to claim 16, wherein the coating agent is in a liquid state at 23° C.

25. The method according to claim 16, wherein the absorbent pad comprises a topsheet, a backsheet and an absorbent core arranged between the topsheet and the backsheet and wherein the method step d) include to incorporate the coated superabsorbent particles in the absorbent core.

26. The method according to claim 25, wherein the absorbent core includes a mixture of cellulose fibers and the coated superabsorbent particles.
